(19) United States
(12) Patent Application Publication
(10) Pub. No.: US 2013/0023790 A1
(43) Pub. Date: Jan. 24, 2013

(54) BIOPSY DEVICE

(71) Applicant: Jeremy R. Schaeffer, Bloomington, IN (US)

(72) Inventor: Jeremy R. Schaeffer, Bloomington, IN (US)

(21) Appl. No.: 13/548,618

(22) Filed: Jul. 13, 2012

Related U.S. Application Data
(60) Provisional application No. 61/509,133, filed on Jul. 19, 2011.

(51) Int. Cl. A61B 10/02

(52) U.S. Cl. 600/567

(57) ABSTRACT

Disclosed is biopsy device that includes a stylet with a sample cavity at the end, a cutting cannula movable over the stylet with a cutting end, a firing mechanism operable to suddenly change the position of the cutting cannula to move the cutting cannula over the sample cavity and a grip from which the stylet and cutting cannula extend, where the grip is held in the palm of one hand with the web of the hand between the index finger and the thumb with the stylet and the cutting cannula oriented along the longitudinal axis of the grip and a trigger on the end of the grip to actuate the firing mechanism, where the trigger is operated by the thumb of the hand gripping the grip to actuate the firing mechanism while the same hand holds the grip in a palm grip.

### REFERENCE TO RELATED APPLICATION

This application claims the benefit of U.S. Provisional Application No. 61/509,133, filed Jul. 19, 2011, entitled BIOPSY DEVICE, which is hereby incorporated by reference.

### BACKGROUND

A biopsy is a medical test involving the removal of cells or tissues for examination. It is the medical removal of tissue from a living subject to determine the presence or extent of a disease. The tissue is generally examined under a microscope by a pathologist, and can also be analyzed chemically. For example, biopsies can be useful in diagnosing various forms of cancer and other diseases affecting tissue.

A variety of biopsy needles and similar devices for obtaining a tissue sample from a patient are known. Commonly, such devices include a thin needle or stylet that can be inserted into the skin of the patient near the location of tissue to be sampled, such as suspected malignancies or other tissue of interest. Once the distal end or other cutting portion of the needle or stylet is within the tissue of interest, a portion of the tissue is excised and captured. The needle or stylet is withdrawn with the tissue sample, which can be retrieved from the device and studied.

Such products have proven quite effective in obtaining tissue in a minimally-invasive manner and with minimal discomfort to the patient. Their lightweight, low-cost nature, combined with their ease of operation and reusability, make them excellent for sampling tissue that may present or indicate a health problem to the patient.

A core needle biopsy is a percutaneous (“through the skin”) procedure that involves removing small samples of tissue using a hollow “core” needle. For palpable lesions (those able to be felt through the skin), this is accomplished by fixing the lesion with one hand and performing a freehand needle biopsy with the other.

In the case of non-palpable lesions (those unable to be felt), stereotactic x-ray or ultrasound image guidance is used. Stereotactic x-ray uses computers to pinpoint the exact location of a mass based on x-rays taken from two different angles. The computer coordinates will help the physician to guide the needle to the correct area to be biopsied. With ultrasound, the radiologist or surgeon will watch the needle on the ultrasound monitor to help guide it to the area of concern. This often requires the physician to operate the imaging equipment with one hand while manipulating the biopsy needle with the other.

### SUMMARY

Disclosed in one aspect is a biopsy device that includes a stylet with a sample cavity at the distal end, a cutting cannula movable over the stylet with a cutting end on the distal end, a firing mechanism operable to suddenly change the position of the cutting cannula to move the cutting cannula over the sample cavity, and a grip with a longitudinal axis, a proximal end and a distal end from which the stylet and cutting cannula extend, where the grip is constructed and arranged to be held and secured in the palm of one hand, with the web of the hand between the index finger and the thumb at the proximate end of the grip with the stylet and the cutting cannula oriented with the longitudinal axis and a trigger on the proximal end of the grip operable to actuate the firing mechanism, where the trigger is constructed and arranged to be operated by the thumb of the hand gripping the grip to actuate the firing mechanism while the same hand holds and secures the grip in a palm grip.

A second disclosed aspect is a method of using the biopsy device including grasping the grip of the biopsy device with the palm and fingers of a single hand with the thumb of the single hand positioned over the trigger on the proximal end of the grip and with the web of the hand between the index finger and the thumb positioned at the proximate end of the grip, inserting the sample cavity into a biopsy site and then, while grasping the grip with the palm and finger of the single hand in a palm grip to secure the position of the sample cavity in the biopsy site, actuating the trigger with the thumb of the single hand thereby activating the firing mechanism and advancing the cutting cannula over the sample cavity thereby severing a sample of tissue from the biopsy site in the sample cavity.

### BRIEF DESCRIPTION OF THE DRAWINGS

FIG. 1 is top plan view of a prior art biopsy device.

FIG. 2 is a top plan view of a biopsy device.

FIG. 3 is an elevational view of the FIG. 2 biopsy device.

FIG. 4 is a bottom view of the FIG. 2 biopsy device.

FIG. 5 is an exploded perspective assembly view of the FIG. 2 biopsy device.

FIG. 6 is a front perspective view of the FIG. 2 biopsy device gripped in an operator's hand in a palm grip.

FIG. 7 is a back perspective view of the FIG. 2 biopsy device gripped in an operator's hand in a palm grip.

FIG. 8 is a side perspective view of the FIG. 2 biopsy device gripped in an operator's hand in a palm grip.

### DETAILED DESCRIPTION

Reference will now be made to certain embodiments and specific language will be used to describe the same. It will nevertheless be understood that no limitation of the scope of this disclosure and the claims is thereby intended, such alterations, further modifications and further applications of the principles described herein being contemplated as would normally occur to one skilled in the art to which this disclosure relates. In several figures, where there are the same or similar elements, those elements are designated with the same or similar reference numerals.

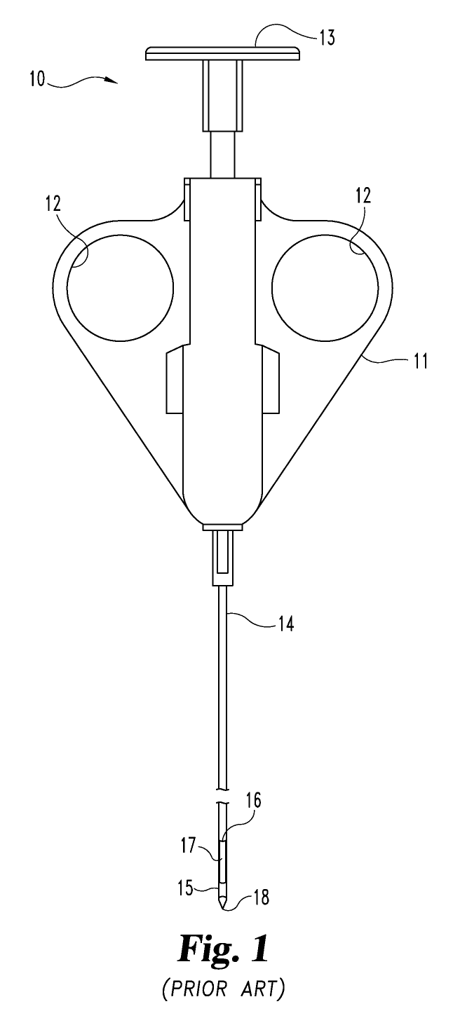

Referring to FIG. 1, a prior art biopsy device 10 is illustrated including handle 11, outer cannula 14 and sampling member 15. Handle 11 includes finger grips 12 and trigger 13. Outer cannula 14 includes cutting tip 16 on the distal end. Sampling member 15 includes sample cavity 17 and tip 18. Depressing trigger 13 actuates a mechanism in handle 11 that rapidly moves outer cannula 14 and cutting tip 16 over sample cavity 17 to cut a biopsy sample that is retained in sample cavity 17 inside of outer cannula 14.

Applicants have identified that in some cases physicians or other users may have difficulty taking precise biopsy samples with biopsy device 10. Biopsy device 10 is constructed to be actuated by depressing trigger 13 with the operator's thumb while gripping device 10 with the index and middle finger through finger grips 12. This grip configuration requires the index and middle fingers to maintain the relative position of biopsy device 10 while the thumb depresses trigger 13. Actuating trigger 13 requires application of a counteracting force to finger grips 12, opposite the force applied to trigger 13. Applicants have identified that, once device 10 is advanced to the proper location to retrieve a sample, a user may pull handle 11 toward trigger 13 when depressing trigger 13 to actuate the mechanism. This can move the location of sample cavity 17 at the moment the mechanism is activated to take the biopsy sample, which can result in the biopsy sample being taken from the wrong or a less-desirable location.

Physicians using biopsy device 10 often have only a single hand available to manipulate device 10 because their other hand is busy with other tasks, for example, fixing or palpating the lesion with one hand or operating an imaging device such as an ultrasound to locate sample cavity 17. Because of this, biopsy devices should be operable with a single hand to insert the biopsy device, stabilize the location of the sample cavity and to actuate the sampling mechanism.

The biopsy device described herein addresses this problem by providing an improved grip configuration that allows the physician to use a palm grip instead of a finger grip when using the biopsy device.

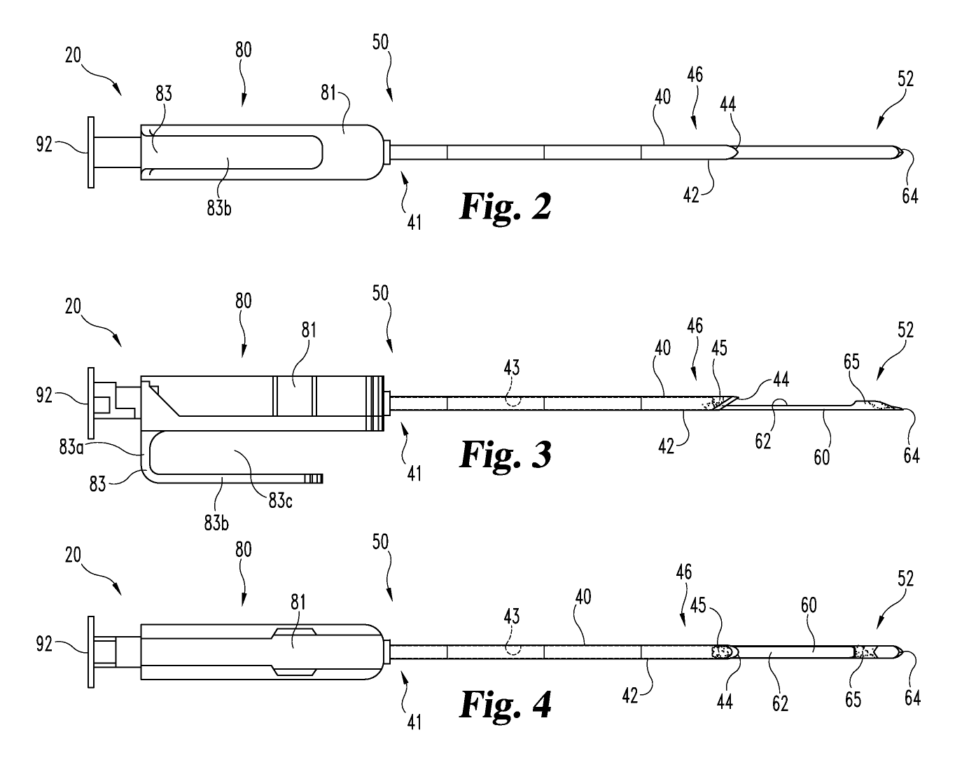

Referring to FIGS. 2-4, biopsy device 20 is illustrated including cannula 40, sampling member 60 and housing 80. Biopsy device 20 is shown in a “primed” condition in FIGS. 2-4, as described below. Cannula 40 includes wall 42, and tip 44. Wall 42 defines lumen 43 between proximal end 41 and distal end 46. Cannula 40 may also optionally include visualization marker 45. In some embodiments, tip 44 is constructed and arranged to penetrate tissue, for example with a cutting edge. In the illustrated embodiment, tip 44 is pointed and bevel sharpened entirely around cannula 40 for cutting tissue.

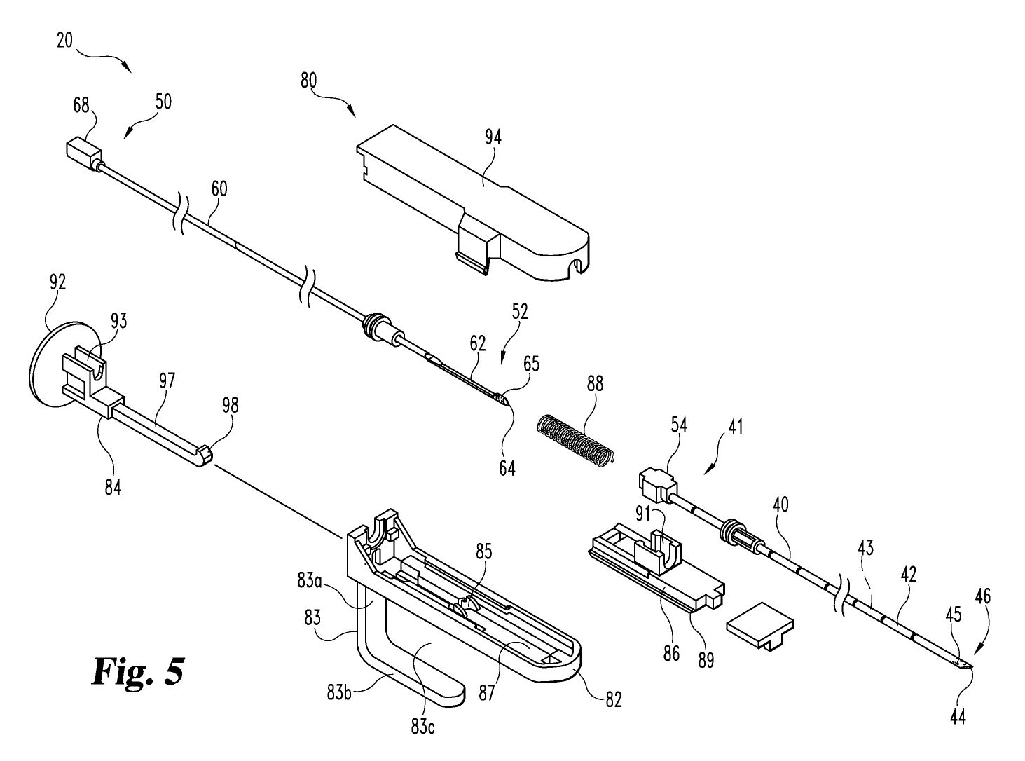

Sampling member 60 includes sample cavity 62, tip 64 at distal end 52, and hub 68 at proximal end 50 (as shown in FIG. 5). Sampling member 60 may also optionally include visualization markers 65. In some embodiments, tip 64 is constructed and arranged to penetrate tissue, for example with a cutting edge. In the illustrated embodiment, tip 64 is pointed and bevel sharpened for cutting tissue. Sampling member 60 can be comprised of any suitable material, such as a plastic and/or metal material. In certain embodiments, sampling member 60 comprises a superelastic alloy, for example, a superelastic Ni—Ti alloy. In other embodiments, sampling member 60 comprises a stainless steel alloy. As shown in FIGS. 2-4, sampling member 60 is disposed in lumen 43 of cannula 40.

As noted above, cannula 40 and/or sampling member 60 may optionally include visualization markers 45 and/or 65 near tips 44 and/or 64 and/or sampling cavity 62 to provide enhanced visualization during insertion and use. For example, when utilizing ultrasonic visualization techniques, visualization markers 45 and/or 65 could comprise an echogenic marker such as a series of small dimple-like indentations on the outer surface of cannula 40 and/or sampling member 60, for example those used on ECHOTIP® Echogenic Needles available from Cook Medical, Bloomington, Ind., USA, to provide enhanced ultrasonic return. In other embodiments, a radiopaque marker, such as a band of bismuth or titanium, could be used to provide enhanced x-ray response during fluoroscopy or other x-ray visualization techniques. Visualization marker 45 and/or 65 may improve the ability of an interventionalist to monitor the position of tips 44 and/or 64 and/or sampling cavity 62 within a patient's body during use.

As assembled, biopsy device 20 includes sampling member 60 disposed in the lumen of cannula 40 with hub 54 engaged with carriage 86 and hub 68 engaged with pad 92. Biopsy device 20 has three positions (uncocked, cocked and primed) which provide two relative positions between cannula 40 and sampling member 60. In the uncocked and cocked positions of the illustrated embodiment, tip 64 protrudes slightly outside of cannula 40 and tip 44 but with sample cavity 62 substantially within or covered by cannula 40. In the primed position, as illustrated in FIGS. 3 and 4, sample member 60 is extended partially outside of cannula 40 and tip 44, exposing some or all of sample cavity 62 outside of cannula 40 and tip 44.

Referring to FIG. 5, cannula 40 can be soldered or otherwise affixed to hub 54 using one of selected well-known techniques, including that of Hall described in U.S. Pat. No. 5,354,623. Cannula 40 can be comprised of any suitable material, such as a plastic and/or metal material. In certain embodiments, cannula 40 comprises a superelastic alloy, for example, nitinol (Ni—Ti alloy), that permits elastic manipulation of cannula 40 beyond the point at which other metals would experience plastic deformation.

Housing 80, as illustrated in FIGS. 2-5, defines grip 81 and includes grip base 82, and grip cover 94 and contains actuator 84, drive carriage 86 and spring 88. Grip base 82 includes resilient latch 85 and channel 87. Grip base 82 may also optionally include lateral extension 83. Actuator 84 includes pad 92, opening 93, projection 97 and end boss 98. Drive carriage 86 includes opening 91 and pawl surface 89 (e.g., a tooth, prong and/or groove edge).

The illustrated embodiment of housing 80, lateral extension 83 features a support portion 83a that extends laterally (e.g. substantially perpendicular to the central longitudinal axis of grip 81) and a guard portion 83b that extends longitudinally. Housing 80, lateral extension 83a and guard portion 83b define a hand or finger space 83c guard portion 83b and grip 81, and closed at one end by support portion 83a. Support portions 83a and guard portion 83b assist the user in proper grip and/or orientation of device 20 relative to the user's hand

Cannula hub 54 is received in opening 91 and is coupled to drive carriage 86. Hub 68 is received in opening 93 and is coupled to actuator 84. When assembled, spring 88 is positioned between grip base 82 and drive carriage 86 with sampling member 60 passing through spring 88. Drive carriage 86 is slidable forward and backward (proximally and distally) in channel 87. Catch 85 is positioned in channel 87 to interact with pawl surface 89 to secure the relative position of drive carriage 86 in channel 87.

Biopsy device 20 is “cocked” by pulling pad 92 (and actuator 84) away from grip base 82. End boss 98 on projection 97 interfaces with drive carriage 86 so that when actuator 84 is pulled proximally away from grip base 82, drive carriage 86 is drawn proximally thereby compressing spring 88 between grip base 82 and drive carriage 86. At the same time, as actuator 84 draws drive carriage 86 proximally, both cannula 40 and sampling member 60 are moved proximally. Grip base 82 includes resilient latch 85 that engages and holds pawl surface 89 on the underside of drive carriage 86, thereby holding drive carriage 86 in the cocked position. This may be done before biopsy device 20 is inserted into flesh.

After cocking, biopsy device 20 is “primed” by moving actuator 84 distally, i.e., toward grip base 82. This extends sampling member 60 partially outside of cannula 40 thereby exposing sample cavity 62 to the tissue to be biopsied. Priming is normally done after biopsy device 20 is inserted proximate to the desired biopsy location. Note that priming biopsy device 20 requires moving actuator 84 only partway toward grip base 82. Biopsy device 20 may provide tactile feedback to the operator approximately at the point it is fully primed and/or ready for firing.

Spring 88 can be subsequently released by pushing pad 92 the rest of its range or movement toward grip base 82 generating distally directed movement of actuator 84 such that projection 97 engages resilient latch 85, causing resilient latch 85 to disengage from pawl surface 89, releasing drive carriage 86 and allowing spring 88 to move drive carriage 86 (and cannula 40) proximally which rapidly moves cannula 40 over sample cavity 62 of sampling member 60. A suitable firing mechanism is disclosed in U.S. Pat. No. 6,056,760 to Koike. (The mention of this firing mechanism is not intended to be limiting or restrictive, but is simply provided as an example of a suitable firing mechanism.)

Actuation of the firing mechanism in housing 80 propels cannula 40 over sampling member 60 to sever and trap tissue within sample cavity 62 of the sampling member 60. The disclosed firing mechanism is a single action biopsy device which is effective when used to obtain tissue samples. Alternative embodiments can, for example, utilize a double-action firing device such as the device disclosed in U.S. Pat. No. 5,538,010 to Darr, which may be used in place of the single-action mechanism disclosed herein.

When assembled together, grip base 82 and grip cover 94 define housing 80 and grip 81. Grip cover 94 fits onto grip base 82, covering and protecting spring 88 and sliding engagement between drive carriage 86 and grip base 82 from debris and interference. Grip 81 defines a grip circumference between approximately 9 cm and approximately 15 cm and grip 81 defines a maximum grip span between approximately 2 cm and approximately 5 cm. As detailed herein, grip 81 is configured and arranged to allow manipulation of pad 92 with the thumb to release cocked spring 88 while gripped in a palm grip by a single hand.

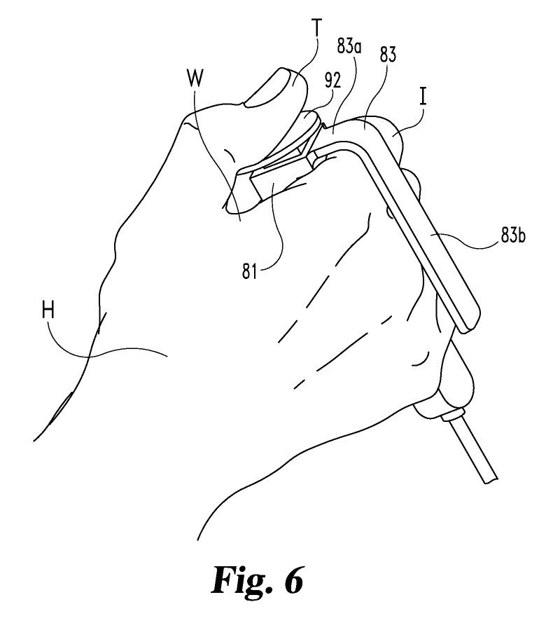

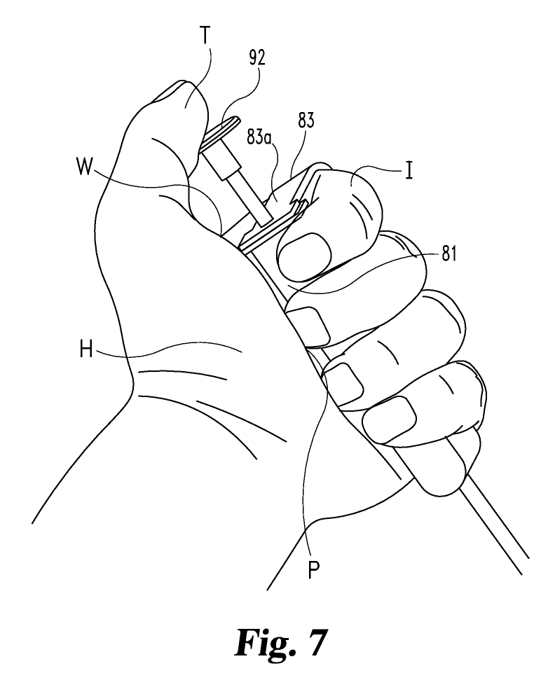

As shown in FIGS. 6 and 7, grip 81 is constructed and arranged to be secured in palm P of a single hand H in a palm grip with web W of that single hand between index finger I and thumb T positioned toward the proximate end of grip 81 with pad 92 depressible distally toward housing 80 by thumb T. If included, lateral extension 83 may be positioned against index finger I (as illustrated) or web W as desired. Lateral extension 83 may also be configured to partially wrap around hand H. With this grip, thumb T can depress and move pad 92 distally toward housing 80 substantially without a natural inclination to move the position of hand H in opposition to the movement of thumb T. In the illustrated embodiment, practically all of the user's hand (save his or her thumb T) is below or distal of pad 92. That placement generally reduces the length of the hand/biopsy device combination compared to a handle in which the user's hand extends away from the handle, and that reduction provides better stability and control for some users.

Once cocked, e.g. as described above, biopsy device 20 can be used by grasping grip 81 with the palm and fingers of a single hand H with thumb T positioned over pad 92 on proximal end 50 with web W of hand H positioned at the proximate end of the grip. Held in this way, the physician can insert biopsy device 20 into a patient so that a portion of device 20 (e.g. cannula 40 and/or sampling member 60) is in or adjacent to a desired biopsy site. While grasping the grip with palm P and fingers of hand H to secure the position of sample cavity 62 in the biopsy site, thumb T can first move pad 92 distally toward housing 80, extending sampling member 60 into or through tissue to be sampled. After sample cavity 62 is fully exposed, the physician can then move pad 92 additionally toward housing 80 to overcome catch 85 and release cannula 40 to rapidly move cutting tip 44 over sample cavity 62 to sever and retain a sample of tissue from the biopsy site in the same cavity. In the illustrated embodiment, thumb T actuates pad 92 by pushing against pad 92 and moving pad 92 in a distal direction toward grip 81 along the longitudinal axis of biopsy device 20.

In the example of FIGS. 6-7, the user inserts his or her hand into space 83c, with the side of the index finger adjacent or abutting the inside of support portion 83a and the tops of at least the index finger (and in the illustrated embodiment, all of the other fingers) adjacent or abutting the inside of guard portion 83b. In that embodiment, support portion 83a provides a buttress or counteraction to the pressing action by the user's thumb. As pad 92 is pressed by thumb T, support portion 83a can engage the user's finger(s). With such engagement and/or the friction from the hold of the user's hand on grip 81, housing 80 remains substantially stationary during firing, without any affirmative pulling force from the hand on housing 80. Guard portion 83b may provide assurance that the user is correctly holding device 20, as well as control over the device and security by limiting or eliminating the chance for grip 81 to slip from the user's hand.

It will be seen that in the illustrated embodiment grip 81 is generally elongated and along a central axis parallel or identical to a central longitudinal axis of cannula 40 and/or sampling member 60. Grip 81 is generally rectangular (in cross section) in the illustrated embodiment, and as indicated above, is of a length to fit across the palm of a user's hand with the user's thumb extendable over pad 92. Other configurations of grip 81 may be used, such as a substantially cylindrical solid, or with ergonomic section(s) that accommodate the user's fingers relatively snugly.

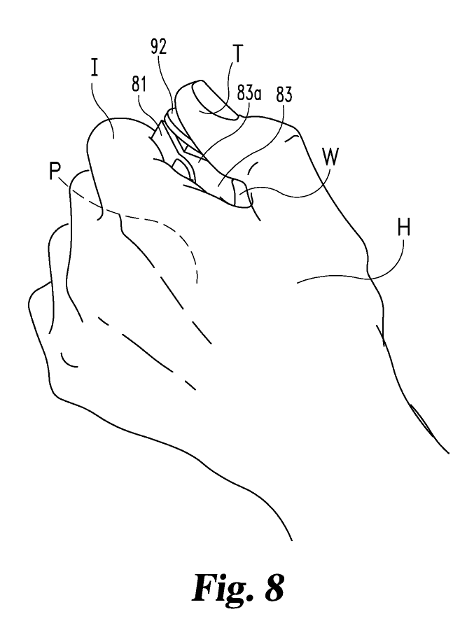

As shown in FIG. 8, grip 81 may also be constructed and arranged so that lateral extension 83 may be alternatively secured in palm P of a single hand H in a palm rather than extending over index finger I and partially wrapping around hand H as shown in FIGS. 6 and 7. Utilizing lateral extension 83 in this way may optionally provide a wider grip width and grip circumference to accommodate larger hands. Once again, with this grip, thumb T can depress and move pad 92 distally toward housing 80 substantially without a natural inclination to move the position of hand H in opposition to the movement of thumb T.

While the disclosure has been illustrated and described in detail in the drawings and foregoing description, the same is to be considered as illustrative and not restrictive in character, it being understood that only certain specific embodiments have been shown and that all changes and modifications that come within the spirit of the disclosure are desired to be protected.

I claim:

1. A biopsy device comprising:
a stylet comprising a sample cavity adjacent a distal end;
a cutting cannula comprising a distal cutting end, wherein the cutting cannula is movable over the stylet;
a firing mechanism operable to suddenly change the position of the cutting cannula to selectively move the cutting cannula over the sample cavity;
a grip having a proximal end and a distal end from which the stylet and cutting cannula extend, wherein the grip defines a longitudinal axis, wherein the grip is constructed and arranged to be held and secured in the palm of a single hand of an operator in a palm grip with the web of the single hand between the index finger and the thumb positioned proximate to the proximate end of the grip and wherein the stylet and the cutting cannula are oriented along the longitudinal axis; and
a trigger on the proximal end of the grip operable to actuate the firing mechanism, wherein the trigger is constructed and arranged to be operable by the thumb of the single hand gripping the grip to actuate the firing mechanism while the single hand holds and secures the grip in the palm of the single hand.

2. The biopsy device of claim 1 further comprising a lateral extension on the proximal end of the grip constructed and arranged to interface with the index finger of the single hand of the operator to resist longitudinal movement of the biopsy device in the distal direction with respect to the single hand of the operator when actuating the plunger.

3. The biopsy device of claim 1, wherein the trigger actuates the firing mechanism by movement of the trigger along the longitudinal axis.

4. The biopsy device of claim 3, wherein the trigger includes a pressure surface constructed and arranged for the thumb of the single hand of the operator to apply pressure to the trigger in a distal direction along the longitudinal axis to actuate the firing mechanism.

5. The biopsy device of claim 1, further comprising a mechanical potential releasable by actuating the trigger to suddenly change the position of the cutting cannula to selectively cover the sample cavity.

6. The biopsy device of claim 1, wherein the firing mechanism is positioned within the grip.

7. The biopsy device of claim 1, wherein the grip has a grip circumference between approximately 9 cm and approximately 15 cm.

8. The biopsy device of claim 1, wherein the grip has a maximum grip span between approximately 2 cm and approximately 5 cm.

9. A method comprising:
grasping a grip of a biopsy device with the palm and fingers of a single hand with the thumb of the single hand positioned over a trigger on the proximal end of the grip and with the web of the hand between the index finger and the thumb positioned at the proximate end of the grip, wherein the grip is operatively coupled to a stylet including a sample cavity, wherein the grip contains a firing mechanism operatively coupled to a cutting cannula including a distal cutting end positioned around the stylet, wherein the trigger is constructed and arranged to selectively activate the firing mechanism to advance the cutting cannula over the sample cavity with sufficient speed to sever a sample of tissue located in the sample cavity and wherein the stylet and the cutting cannula are oriented along a longitudinal axis of the grip;
inserting the sample cavity into a biopsy site; and
while grasping the grip with the palm and finger of the single hand in a palm grip to secure the position of the sample cavity in the biopsy site, actuating the trigger with the thumb of the single hand thereby activating the firing mechanism and advancing the cutting cannula over the sample cavity thereby severing a sample of tissue from the biopsy site in the sample cavity.

10. The method of claim 9, further comprising positioning the index finger of the single hand against a lateral extension on the proximal end of the grip to resist longitudinal movement of the grip with respect to the single hand when actuating the trigger with the thumb of the single hand.

11. The method of claim 9, further comprising moving the trigger in along the longitudinal axis of the grip to actuate the trigger.

12. The method of claim 9, further comprising, pushing against a pressure surface on trigger in a distal direction along a longitudinal axis of the grip with the thumb of the single hand to actuate the trigger.

13. The method of claim 9, wherein the firing mechanism is a mechanical potential releasable by the trigger.

14. The biopsy device of claim 9, wherein the grip has a grip circumference between approximately 9 cm and approximately 15 cm.

15. The biopsy device of claim 9, wherein the grip has a maximum grip span between approximately 2 cm and approximately 5 cm.
